(19) United States
(12) Patent Application Publication
(10) Pub. No.: US 2013/0035611 A1
(43) Pub. Date: Feb. 7, 2013

(54) Vaginal Bio-mechanics Analyzer

(71) Applicant: Carter Bell White, Mesquite, TX (US)

(72) Inventor: Carter Bell White, Mesquite, TX (US)

(21) Appl. No.: 13/564,682

(22) Filed: Aug. 1, 2012

Related U.S. Application Data
(60) Provisional application No. 61/574,290, filed on Aug. 1, 2011.

(51) Int. Cl. A61B 5/103

(52) U.S. Cl. 600/591

(57) ABSTRACT

The present invention relates to a method for measuring the elasticity of the anterior walls of the human vagina. The elasticity of the vagina walls degrade as women age. When a condition occurs called pelvic organ prolapse, the vaginal walls have lost much of the visco-elastic properties. The ability to measure the elasticity in healthy women at an early age and track the changes over time will give researchers the chance to develop new therapies to manage this growing problem. The present invention makes multiple data measurements of vacuum pressures and proximity measurement, by the use of a small insertable, user friendly and quickly sterilizable vaginal device. The proximity sensor not only measures the deformation of the skin pulled into a small hole of the vaginal probe but also measures the skin deformation after the skin has retracted out of the probe hole.

### REFERENCE TO RELATED APPLICATION

This application claims the benefit of 35 U.S.C. 111(b) Provisional Patent Application Ser. No. 61/574,290 which was filed on Aug. 1, 2011 and entitled “Electronic Skin Elasticity Meter”.

### FIELD OF THE INVENTION

The present invention relates to an apparatus that measures the elasticity of skin. Skin elasticity is measured to determine the effects of medications, skin creams, surgery procedures and the effects of aging. The present invention is designed to measure the skin elasticity of the inner walls of the vagina to detect changes in the integrity of connective tissues in the vagina. The present includes a small probe that allows a physician to easily perform elasticity measurements on patients during a regular office exam. The present invention provides the physician with a medical device to determine, among other conditions, if a woman is susceptible to prolapse, a condition that happens when the bladder falls down into the vagina.

Skin elasticity is calculated from the data derived from the combination of vacuum pressure, time, the amount of skin pulled by the vacuum, the length of time the skin returns to the original shape, and the recoil reaction of the skin. The values are collected, calculated, and stored by the microcontroller and then down loaded to a computer through a data port or USB port. The data can be compiled by a computer program to display tables, plot graphs, indicate changes in the vaginal wall elasticity and assist physicians diagnose any change of elasticity and the probability of prolapse and other conditions related to vaginal disease.

The present invention is not limited to the vagina skin elasticity measurement. The present invention can test elasticity of any skin on any area of the body of any living animal. The present invention will also test the elasticity of flexible materials such as rubber, vinyl, foams or other elastic materials

### BACKGROUND OF THE INVENTION

The present invention relates to an electro mechanical device that measures skin elasticity for assessing the viscoelastic properties of the anterior wall of the vagina. Vaginal wall tissue deterioration can cause pelvic organ prolapse (POP), a hernia of the pelvic organs to or through the vaginal opening. POP affects a large number of aging women that often necessitates surgical repair and tends to recur over time. Approximately 200,000 operations are performed yearly in the United States for POP. Although not life threatening, POP is life altering and results in significant quality of life changes in women.

Medical researchers have studied vaginal wall properties in fresh excised tissue, at the time of surgery, using an Instron tensile testing machine but this is limited by its applicability, namely patients requiring surgery. Currently, evaluation of the vaginal wall is limited to physical examination and imaging modalities. There are no quantitative and practical devices that a physician can use during an office visit to measure the unique viscoelastic properties of the vagina to objectively determine tissue deterioration. The ability to measure the elasticity of the inner walls of the vagina in healthy patients for study controls, patients in less advanced degrees of POP, patients before and after surgical repair and patients on hormonal therapy will lead to a myriad of common vaginal interventions, from pelvic floor therapy to reconstructive surgery. Like the thermometer to determine how sick a patient is, the present invention will serve as a diagnostic resource for clinicians and researchers interested in the management of POP.

Skin elasticity measurement devices that were found in the patent search include US2008/0234607 A1. It applies a vacuum to a chamber that is placed over an area of the skin. When the vacuum draws the skin through an opening a video camera in an adjacent chamber captures light reflected from the skin. U.S. Pat. No. 7,955,278 B1 creates a vacuum that draws the skin into a chamber until the skin reaches the vacuum tube in the chamber. The vacuum pressures are measured and pressure changes are used to calculate elasticity. U.S. Pat. No. 5,278,776, describes the use of a camera that monitors the movement of dots placed on the skin. When the vacuum is applied the skin moves into the chamber causing the dots to move. The elasticity is determined by the dot separation.

Prior art is designed to test the elasticity of skin on the surface of the body. The present invention is a safe, easily insertable, user-friendly, and quickly sterilizable vaginal device that would allow rapid and reproducible measurements of different areas of the vagina, in the office setting. The present invention is simple to use but extremely accurate. The probe design is small enough to be inserted in the vagina, yet measure precisely the tissue deflection and recovery under mild suction and vacuum release. The stored data for each patient can be compared to previously collected data to detect the changes in tissue elasticity. For the first time, the present invention allows for a direct in-vivo measurement of vaginal wall tissue properties.

### OBJECT OF THE INVENTION

The present invention is used as a medical device to give physicians data to diagnose, predict and repair various conditions associated with skin due ageing or disease. The present invention is a small and portable unit consisting of a vacuum canister, a vacuum pump, an electronic control unit with a liquid crystal display, and an elasticity measuring wand assembly. The wand, FIGS. 1 and 2, is a hollow oval tube approximately six inches in length by approximately three quarters of an inch wide. A 10 millimeter hole is located on the edge of the wand approximately one half inch from the sealed end. The wand handle FIG. 2, has a circuit board that contains an electronic proximity sensor and connections for the data cable and vacuum line. When the handle is attached to the wand the sensor is positioned under the 10 millimeter hole. The wand assembly is inserted into the vagina to measure the elasticity of the anterior walls of the vagina. A vacuum is applied to the wand assembly making a seal through the hole in the wand. The vacuum causes the skin to be pulled into the hole of the wand assembly. When the vacuum reaches a preset level, the microcontroller reads the proximity sensor values. The proximity sensor measures the distance from the proximity sensor to the skin pulled into the hole. The microcontroller computes the distance measured to determine how much skin was pulled in from the vacuum. Skin elasticity is calculated from the data derived from the combination of vacuum pressure, time, the amount of skin pulled in by the vacuum, and the shape of the curve plotted from the data. The values are calculated and stored by the microcontroller and are transferred to a computer through a data port. The data is compiled and stored in a program that plots graphs for the physician to analyze.

### SUMMARY OF THE INVENTION

The present invention is a medical device that analyzes skin elasticity of the inner walls of the vagina. The Vaginal Bio-Mechanics Analyzer is a small and portable unit consisting of a vacuum canister, a vacuum pump, an electronic control unit with a liquid crystal display, remote computer connection, and an elasticity measuring wand assembly depicted in FIG. 3. The wand assembly consists of two parts, the probe FIG. 1 and the handle, FIG. 2. The probe is removable from the handle for sterilization before each patient exam. The probe of FIG. 1 is a hollow tube approximately six inches in length by approximately three quarters of an inch wide in an oval shape with a hole located on the wide edge of the probe and approximately three quarters of an inch from the rounded closed end of the probe. The handle of FIG. 2 has a sensor board that slides into the probe and is positioned precisely beneath the hole in the probe. The vacuum line and data cable in FIG. 3 connect to the handle of FIG. 2. The patient exam begins with the physician inserting the probe into the vagina. A low preprogramed vacuum is applied by the control unit of FIG. 3 causing the skin of the vagina to be pulled into the hole in the probe. As the skin moves into the hole the proximity sensor measures the distance between the sensor and the moving skin. The data representing the skin movement, the changes in vacuum pressure and the increments of time are stored by the microcontroller of FIG. 6 located in the control unit. The data port in FIG. 6 connects to a computer that receives the stored data that is downloaded from the control unit. The physician compiles the data to analyze and store for future comparisons. Graphs can be produced such as FIGS. 7, 8, and 9 in common computer programs that represent a visual representation of the vaginal wall elasticity.

### BRIEF DESCRIPTION OF THE DRAWINGS

FIG. 1 shows a preferred embodiment of the probe that is inserted into the vagina.

FIG. 2 shows a preferred embodiment of the handle which attaches to the probe.

FIG. 3 is an illustration of the components of the present invention.

FIGS. 4A and 4B set forth a flow chart of the control unit of the preferred embodiment.

FIG. 5 is a schematic of pneumatic vacuum system of the present invention.

FIG. 6 is a block diagram of the microcontroller and the electrical components of the present invention.

FIG. 7 is a graph representing the data of a patient with prolapse.

FIG. 8 is a graph representing the data of a patient without prolapse.

FIG. 9 is a graph representing the data of the cheek of a patient's face.

### DETAILED DESCRIPTION OF THE PREFERRED EMBODIMENTS

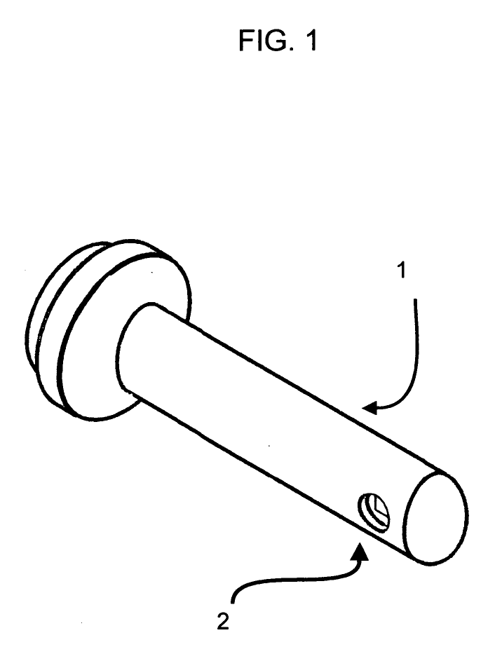

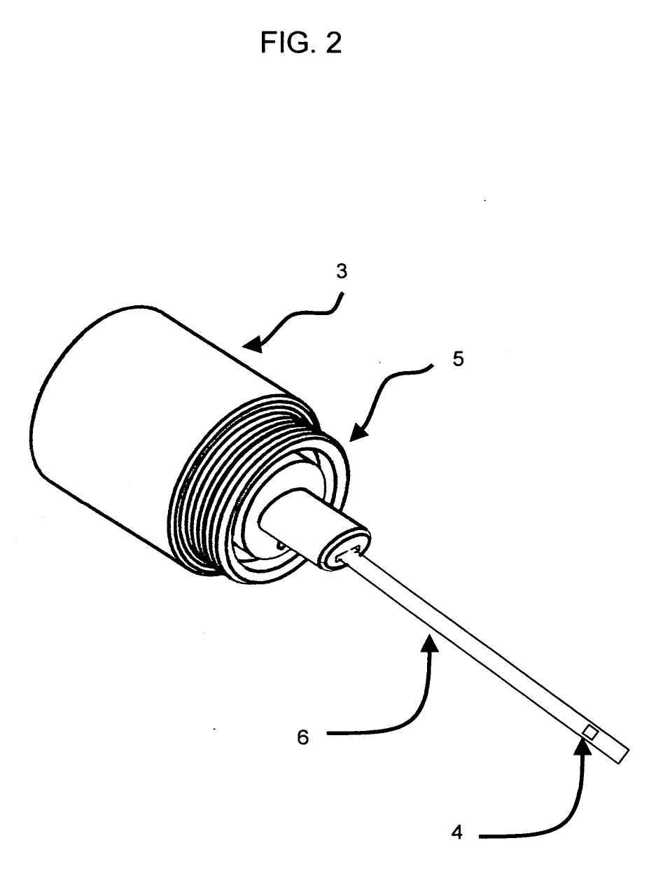

FIG. 1 depicts a hollow tube that is oval. The wide part of the oval is 0.75 inches while the narrow part of the oval is 0.625 inches. The probe 1 is 5.5 inches in length and is the outer part of the wand assembly. A hole 2 has a 10 millimeter diameter and is on the 0.75 inch surface of the probe. Hole 2 centerline is 0.75 inches from the rounded end of the probe 1. The hole 2 allows the skin that is under test to pull down into the hole when a vacuum is applied. As the skin is pulled in the hole, the proximity sensor of FIG. 2 makes measurements as described later here in. The probe 1 has a flange that allows for a vacuum seal when connected to the handle of FIG. 2. The probe 1 is removed from the handle of FIG. 2 to clean and sterilize after use.

FIG. 2 shows the handle 3 with the proximity sensor 4 attached to a circuit board. The handle 3, is 2.75 inches in length and is 2.25 inches in diameter. The probe 1 of FIG. 1 attaches to the handle 3 by the threads 5 to securely hold the probe of FIG. 1 to the handle 3 and make a seal to prevent vacuum leaks. The proximity sensor 4 is precisely positioned beneath the 10 millimeter hole of FIG. 1 when the handle 3 and probe 1 are attached. The sensor circuit board 6 makes an electrical and data connection to the proximity sensor 4.

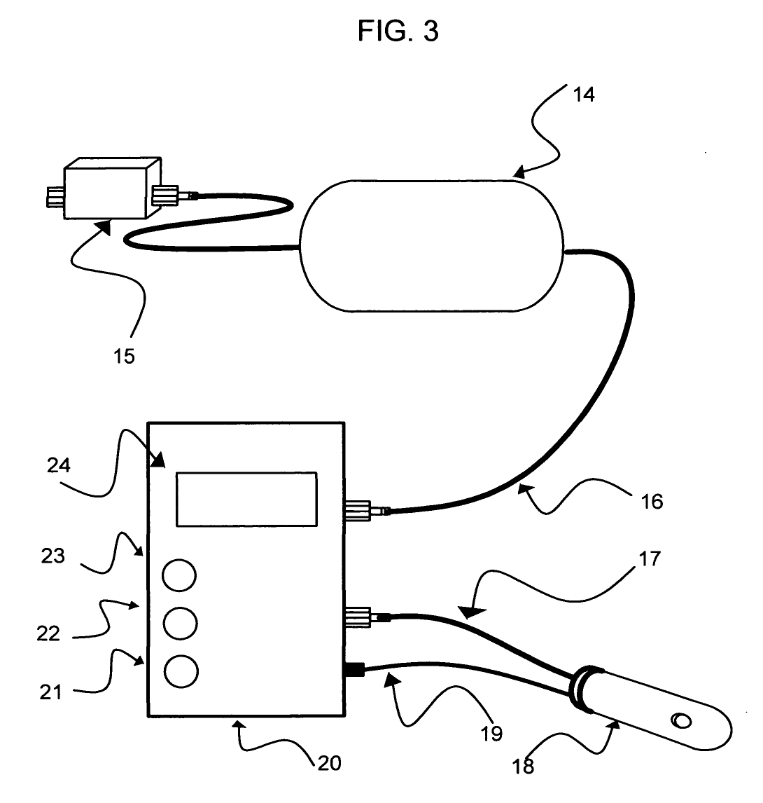

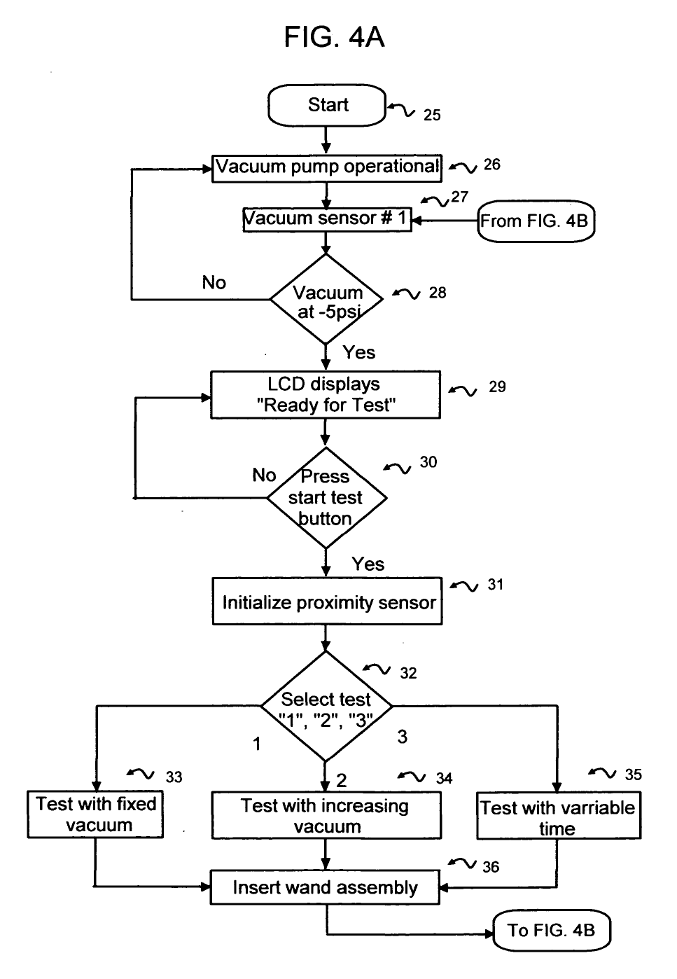

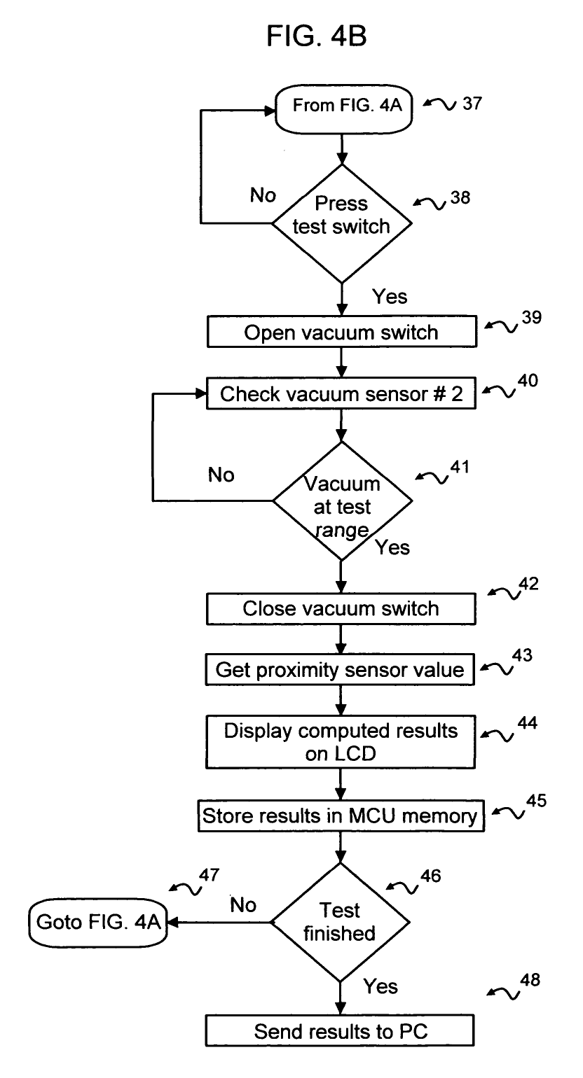

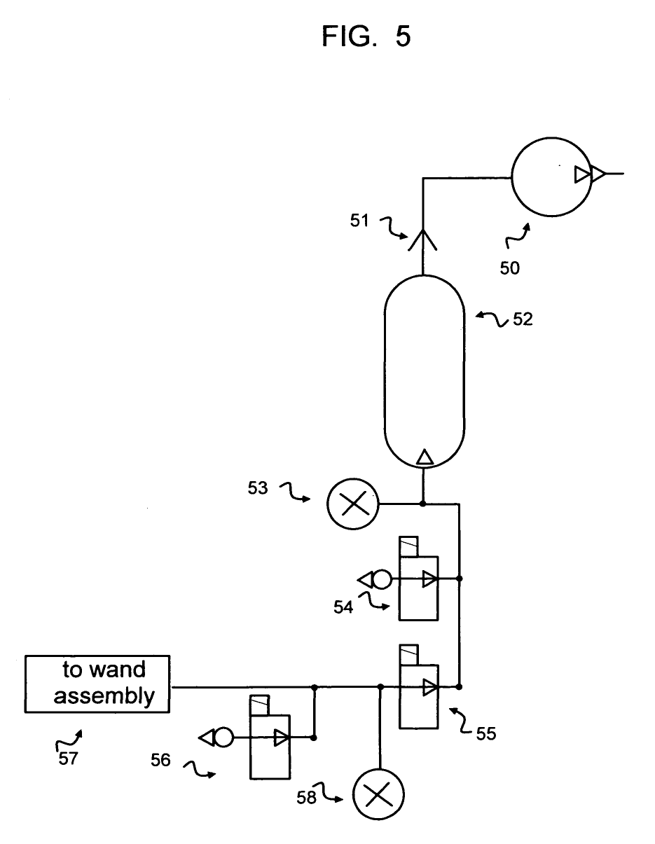

FIG. 3 is an illustration of the components used by the present invention to test skin elasticity. Vacuum canister 14 is evacuated to approximately negative 400 millimeters of mercury by an electrical vacuum pump 15. A vacuum line 16 is connected to the electronic control unit 20. Electronic control unit 20 has a liquid crystal display 24, and switches 21, 22, and 23. The switches 21, 22, 23, and LCD 24 are used to perform menu selections displayed on the LCD screen 24 as described later in FIGS. 4A and 4B. The data cable 19 of wand assembly 18 provides electrical and data connection between the wand assembly and the electronic control unit 20. Data cable 19 is used to transmit serial data from the proximity sensor 4 of FIG. 2 to the microcontroller 62 described later in FIG. 5. Vacuum line 17 is connected to the wand assembly 18 and to the electronic control unit 20. The vacuum line 17 allows a vacuum that is regulated by the control unit 20. The vacuum pump 15 and vacuum storage canister 14 are contained in the control unit 20. The electronic control valves of FIG. 5 are located in the control unit 20.

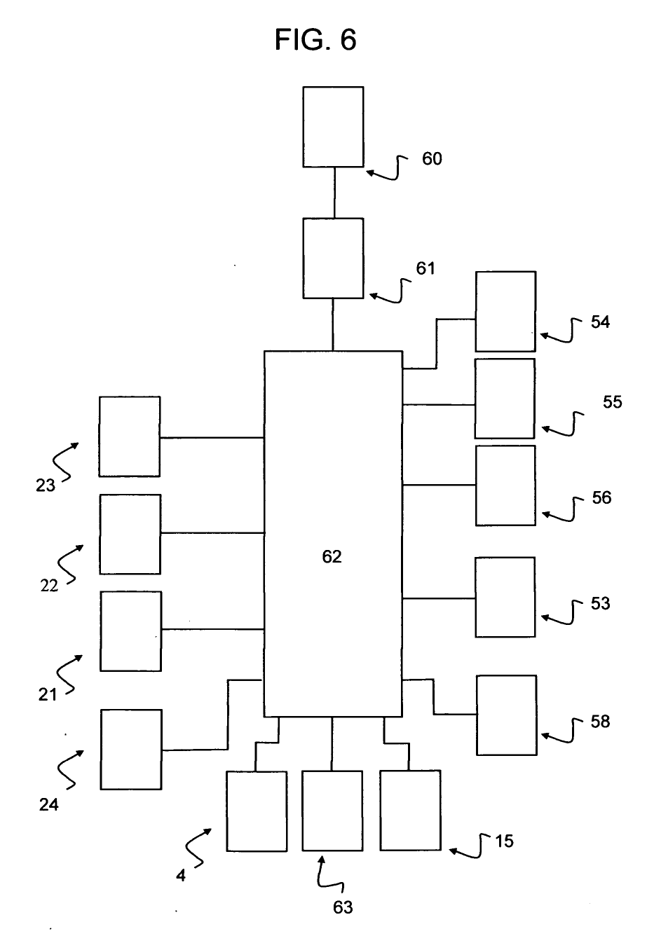

FIGS. 4A and 4B-illustrate a flow diagram of the present invention. The flow diagrams describe only two of a plurality of skin elasticity tests that the present invention can perform. A microcontroller of the present invention is programmed to read and write the control values of the proximity sensor 4 of FIG. 2, the vacuum pump 15, the liquid crystal display of FIG. 3, and the electronic vacuum valves 54, 55 and 56 of FIG. 5. The power is switched on at the step 25, vacuum pump at step 26 is energized and begins aspirating a vacuum in canister 14 of FIG. 3. At step 28, digital vacuum sensor 53 of FIG. 5 outputs an analog signal proportional to the vacuum pressure. The signal is converted to a digital value in the microcontroller. When the vacuum pressure reaches approximately 400 millimeters of mercury at step 28 the vacuum pump 15 of FIG. 3 is switched off. In step 29 the liquid crystal display (LCD) 24 of FIG. 3 displays a message to begin the test. In step 30 the physician presses the start button 23 of FIG. 3. The proximity sensor 4 of FIG. 2 is energized. Internal circuitry in the proximity sensor stabilizes and at step 32 the physician presses select switch 22 of FIG. 3 to choose the type of test to perform. When the test is selected, the physician inserts the wand assembly into the vagina of the patient at step 36. At step 38 the physician presses the test switch 23 of FIG. 3 to start the selected test. At step 39, variable solenoid vacuum valve 55 of FIG. 6 is opened and a vacuum is created in the wand assembly. A small portion the inner wall of the vagina begins to pull into the hole 2 of FIG. 1. At step 41 the vacuum is sensor 58 of FIG. 5 begins sensing the change from atmospheric pressure to a vacuum. At step 41 the variable solenoid valve 55 of FIG. 5 stays open until a predetermined vacuum pressure has been reached and then switched off at step 42. The microcontroller 62 of FIG. 6 receives the proximity value at step 43 from the proximity sensor 4 of FIG. 2. The values are computed and the results are displayed on LCD 24 of FIG. 3 at step 44. At step 45, the results are stored in memory of the microcontroller 62 of FIG. 6. At step 46 the physician is given a choice to start the tests over or at step 48, to download the test results to a computer through the data port 63 of FIG. 6. The type of test decision at step 32 is selected by pressing the switch 22 of FIG. 3. Test one is a test that requires the microcontroller to energize and open the electronic variable valve 56 of FIG. 6 until the vacuum sensor 58 of FIG. 5 senses a preset value. The proximity sensor 4 of FIG. 2 measures the distance and outputs a digital number representing the zero point in distance at zero vacuum. The vacuum sensor 58 of FIG. 5 begins detecting a change of negative pressure while the proximity sensor 4 of FIG. 2 continues to make measurements. The microcontroller 62 of FIG. 6 stores the values from the vacuum sensor 58 of FIG. 5 and proximity sensor 4 of FIG. 2 until the vacuum reaches a predetermined value. The electronic variable valve 56 of FIG. 5 is closed ending the test. The microcontroller 62 of FIG. 6 computes the proximity and vacuum values of the test and stores them in memory for evaluation of skin elasticity. Test 2 at step 34 uses the variable electronic valve 55 of FIG. 6 to vary the vacuum pressure applied to the wand assembly. The test begins at a zero vacuum pressure and for a predetermined time, the vacuum increases to a predetermined level. During the pressure increase, the proximity sensor 4 of FIG. 2 takes a predetermined amount of measurements which are stored in microcontroller 62 of FIG. 6. The sensor 4 of FIG. 2 and vacuum sensor 58 of FIG. 5 values are displayed on the LCD 24 of FIG. 3 at step 44. The values are downloaded to a computer through a data port 63 of FIG. 6 at step 48. The computer compiles the data to plot graphs showing the relationship of vacuum and the increase of skin being pulled through the hole 2 of FIG. 1 vs. time. Test three at step 35 is designed to test the amount of skin pulled through the hole 2 of FIG. 1 while a preset vacuum is held over a change in time. The proximity sensor 4 of FIG. 2 measures the zero vacuum level then stores the value in microcontroller 62 of FIG. 6. The electronic variable vacuum valve 56 of FIG. 5 is opened until a predetermined vacuum pressure is reached. The vacuum in held for a predetermined time. The proximity sensor 4 of FIG. 2, takes a predetermined number of readings that are stored in microcontroller 62 of FIG. 6. The values are downloaded to a computer through data port 63 of FIG. 6 at step 48. The computer compiles the data that can display graphs of time vs. increasing amount of skin pulled through hole 2 of FIG. 1. The present invention is not limited to the described three tests. A plurality of preprogrammed tests are possible to determine skin elasticity.

FIG. 5 is a schematic of the pneumatic vacuum system of the present invention. The vacuum pump 50 is connected by vacuum tubing to a check valve 51 to prevent air from flowing back into the vacuum canister 52 after the vacuum pump is switched off. The vacuum canister 52 is used as a vacuum reservoir for fast evacuation of air through the vacuum system. An electronic vacuum sensor 53 is connected by tubing the vacuum canister 52 and senses the vacuum which outputs an analog voltage proportional to the vacuum pressure. The analog voltage is used by the microcontroller 62 of FIG. 6 to determine the vacuum pressure and keep a constant vacuum pressure in the vacuum canister 52 by switching the vacuum pump 50 on at a preset low value and off for a preset high value. Solenoid pressure valve 54 is connected by tubing to electronic vacuum sensor 53 and is an emergency release valve that is opened to bring the vacuum system to atmospheric pressure. If the vacuum pressure reaches a predetermined level or the physician presses the emergency release switch 21 of FIG. 4, solenoid pressure valve 54 will open to allow the system to come to atmospheric pressure. Electronic variable solenoid valve 55 is connected by tubing to the solenoid valve 54, and restricts the vacuum pressure level that is proportional to the current applied to the solenoid by the microcontroller 62 of FIG. 6. Flow restriction is one of the parameters used in an elasticity test. Solenoid valve 56 is connected by tubing to electronic variable valve 55 and when opened releases the vacuum pressure in the wand assembly. Pressure sensor 58 is connected by tubing to the solenoid valve 56 and sends an analog voltage proportional to the vacuum pressure of the wand assembly to microcontroller 62 of FIG. 6. The microcontroller opens electronic variable valve 55 at the beginning of a test and closes it at a predetermined vacuum level measured by the vacuum sensor 58.

FIG. 6 is a block diagram that illustrates the electronic components of the present invention. Microcontroller 62 is programmed to perform the tasks required to control all the required functions of the flow charts, FIGS. 4A and 4B. The power is preferably a 12 volt direct current power supply 60. The power switch 61 switches on the power to the microcontroller 62. Vacuum solenoid valve 54 is electrically connected to an I/O port that provides power to open the valve to release the vacuum in vacuum canister 14 of FIG. 3. Vacuum sensor 53 is electrically connected to an ND port on microcontroller 62 that reads the analog voltage output from the sensor and converts it to a digital signal used by the microcontroller 62 to switch on and off the vacuum pump 15. Solenoid variable valve 55 is electrically connected to an I/O port that outputs a pulse width modulated signal to vary the current through the solenoid. As the current increases through the valve's solenoid, valve 55 opens wider, allowing more airflow. Solenoid valve 56 is electrically connected to an I/O port on microcontroller 62 and energizes the solenoid at a programmed point to release the vacuum pressure on the wand assembly. Vacuum sensor 58 monitors the vacuum pressure on the vacuum line connected to the wand assembly by outputting an analog voltage to a second A/D input of microcontroller 62. The A/D input converts the analog signal to a digital value proportional to the analog voltage. The microcontroller 62 is programmed to open solenoid valve 56 at a predetermined vacuum pressure. Proximity sensor 4 is electrically connected to an I2C data port on microcontroller 62. Data from proximity sensor 4 is used in the microcontroller 62 to determine the distance from the sensor 4 to the surface of the skin pulled through the hole 2 in FIG. 1 by the vacuum applied. Liquid crystal display 24 is connected to I/O ports to display the various menu options and test results that are computed by the microcontroller 62. Push button switch 23 is electrically connected to microcontroller 62 and when pressed starts the test program to begin collecting data from the pressure sensors 53 and 58 and proximity sensor 4. Push button 22 is electrically connected to microcontroller 62 and when pressed causes the liquid crystal display to display programmed menu choices available for performing the tests. Push button switch 21 is electrically connected to microcontroller 62 and when pressed causes all functions to stop and open the solenoid valve 54, to release the pressure in vacuum canister 14 of FIG. 3 and then open solenoid valve 56 to relieve vacuum pressure on the wand assembly. Data port 63 is electrically connected to microcontroller 62 to allow the data from the microcontroller 62 to transfer to a computer that is programmed to compute, graph and store the skin elasticity data for analysis.

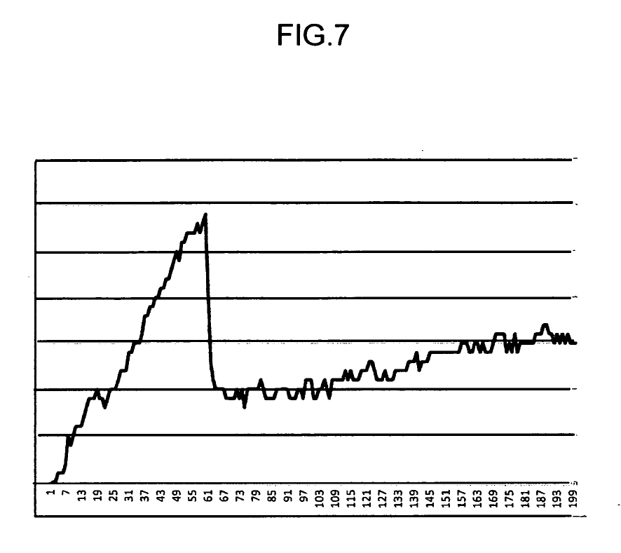

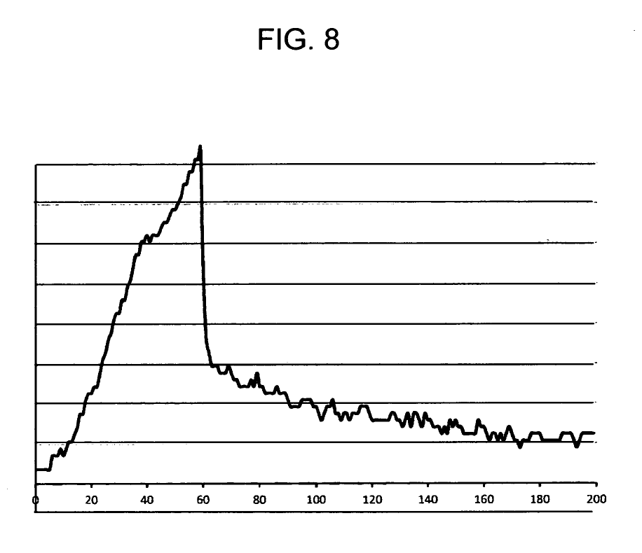

FIG. 7 is a graph of the deformation of the skin in the anterior wall of the vagina of a patient with prolapse. The probe was inserted 5 centimeters with the hole 2 of FIG. 1 pointing up. The test parameters were selected by choosing a menu displayed on the LCD screen 24 of FIG. 3. The test was set to a 20 second time period. The test parameters consisted of a vacuum linearly increased from 0 to 150 millimeters of mercury over a 6 second period while data measurements were recorded in 1/10 of a second intervals. At the end of 6 seconds the vacuum was released. The data was continuously collected for 14 more seconds. The skin deformed to 2.9 millimeters and dropped to 0.9 millimeters in 2/10 ef a second. Over the last 14 seconds the skin gradually rose to 1.5 millimeters. The chart indicates that the vagina's anterior wall of a prolapsed patient lacked elasticity when compared to the chart of FIG. 8, a patient without prolapse.

FIG. 8 is a graph of the deformation of the skin in the anterior wall of the vagina of a patient without prolapse. The test parameters were the same as in FIG. 7. The vacuum was increased from 0 to 150 millimeters of mercury over 6 seconds while data measurements recorded every 1/10 of a second of the proximity sensor 4 of FIG. 2 and the vacuum measurements from the sensor 58 of FIG. 5. The test continued for 14 seconds longer still collecting data each 1/10 of a second. 200 data points from the proximity sensor and 200 data points from the vacuum sensor were transferred to a computer through the data port 63 of FIG. 6. The plotted data show the elasticity of a patient's vagina without prolapse. The peak deformation at 150 millimeters of mercury was 2.1 millimeters with a relaxation from 0.75 millimeters that continued down to 0.25 millimeters at the end of 20 seconds. The chart indicates the skin deformation and elastic properties are significantly different than the patient with a prolapsed bladder.

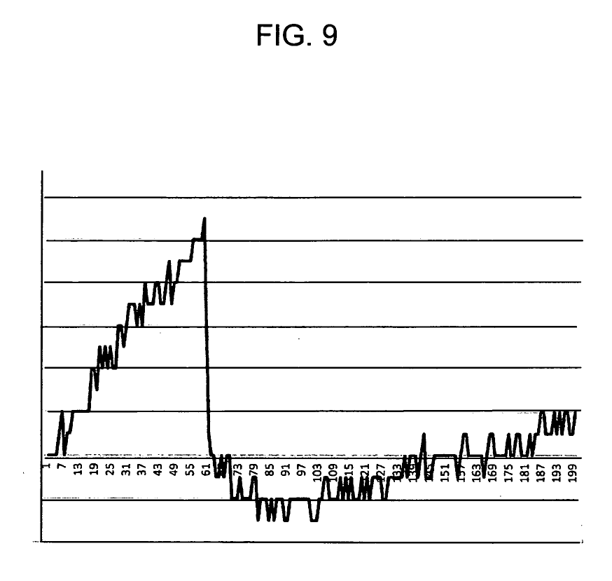

FIG. 9 is a chart of the skin deformation of the cheek on a patient's face. The same parameters and procedures were followed as in FIGS. 7 and 8. The data produced a very different graph that represents the versatility of the present invention. At the peak when the vacuum reached 150 millimeters of mercury the skin deformed to 0.55 millimeters. The vacuum was released and the skin pulled back past zero to −0.15 millimeters. At 14 seconds into the test the skin moved from 0.15 millimeters to 0. Then the skin began moving up until the test was completed at 20 seconds where the skin reached a 0.1 millimeter deflection. The patient under the test was a male approximately 60 year old. The graph indicates the skin bouncing back and passing through zero creating a concave effect on the skin surface. Tests performed on tighter skin surfaces showed a smaller skin deformation but not passing through zero. Another feature the graph depicts is the representation of the patient's heart beat. The groupings of the spikes in the graph at 6 seconds equals to 7 indicating a slightly faster rate of 1 per second. At 20 seconds the groupings of spikes 22 beats or a slightly faster rate than 1 beat per second.

1. A device comprising:
1) a first means of measuring skin elasticity and
2) a second means of recording the deformation of the skin

2. The device of claim 1:
1) Is comprised of a control unit of
a) switches, electronic control valves, and a vacuum pump
b) a microcontroller, pressure sensors and a liquid crystal display
2) a wand assembly
a) that is comprised of a detachable handle and a probe
b) the handle contains a circuit board with a proximity sensor, a data cable connection and a vacuum tube connection
c) and when the probe is attached to the handle a hole in the probe lines up with the proximity sensor.

3. The device of claim 1 stores vacuum data and proximity sensor data to down load to a computer to analyze skin elasticity of the inner walls of the vagina.

4. The probe of the wand assembly
1) is oval in shape 0.75 inches by 0.625 inches allowing the probe to fit comfortably in the human vagina
2) has a means to hold a vacuum while the proximity sensor measures the skin pulled through the hole in the probe
3) and is easily removed from the handle for sterilizing.

5. The proximity sensor is mounted to a circuit board that is attached to the handle of the wand assembly that is
1) positioned on the circuit board to fit under and alien with the hole in the probe when the probe is attached to the handle
2) the proximity sensor measures in hundredths of a millimeter the amount of skin deformed under a vacuum
3) the proximity sensor measures the distance the skin recoils when the vacuum is released
4) the proximity sensor is not limited to measuring the skin inside the hole but continues to measure skin movement outside the hole
